(19) United States
(12) Patent Application Publication
(10) Pub. No.: US 2014/0065221 A1
(43) Pub. Date: Mar. 6, 2014

(54) COMPOSITIONS COMPRISING A FILLER PRODUCT AND AT LEAST ONE BIORESORBABLE AND BIODEGRADABLE SILICA-BASED MATERIAL

(71) Applicants: Rosy Eloy, Ternay (FR); Anders Karlsson, Storvreta (SE)

(72) Inventors: Rosy Eloy, Ternay (FR); Anders Karlsson, Storvreta (SE)

(73) Assignee: GALDERMA RESEARCH & DEVELOPMENT, Biot (FR)

(21) Appl. No.: 14/006,745

(22) PCT Filed: Apr. 2, 2012
(86) PCT No.: PCT/EP2012/055979

§ 371 Date: Nov. 12, 2013

Related U.S. Application Data
(60) Provisional application No. 61/470,211, filed on Mar. 31, 2011.

(51) Int. Cl. A61K 9/14; A61K 47/34; A61K 47/36

(52) U.S. Cl. CPC A61K 9/14; A61K 47/36; A61K 47/34
USPC 424/489; 514/777; 514/626

(57) ABSTRACT

A combination product including at least one filler product and at least at least one bioresorbable and biodegradable silica-based material, for treating incontinence or treating aging of the skin and scars. Also, a composition including, in a physiologically acceptable medium, at least one filler product and at least at least one bioresorbable and biodegradable silica-based material.

The present invention relates to compositions for parenteral application, comprising, in a physiologically acceptable medium, at least one filler product and at least one bioresorbable and biodegradable silica-based material. The invention also relates to the use of these compositions for tissue augmentation, for both therapeutical and cosmetical purposes. Preferentially, the composition is used for treating incontinence, or aging of the skin, in particular for treatment of wrinkles and fine lines, fibroblast depletions, skin dehydration and scars of all types.

Compositions for use in tissue augmentation are desirable for both therapeutical and cosmetical purposes.

A therapeutical application is, for example, augmentation of tissues that need to be enlarged for proper function. Examples of such are the vocal cords, the oesophagus, various sphincters that have become weakened or have too thin tissue mass. The augmentation material is especially suitable for mammary implants but also for urethral or rectal sphincters augmentation, for treatment of faecal or urinary incontinence,

Incontinence can be caused by urinary reflux. Vesicoureteral reflux (VUR) is a malformation of the urinary bladder that affects children and which can give severe urinary tract infections and even irreversible kidney damage. Few alternatives to surgery exist to treat VUR. Deflux®, sold by Oceana Therapeutics is one of them, being used in endoscopic injections. Deflux® was the first hyaluronic acid based product for the treatment of VUR and replaced less attractive alternatives such as Teflon. The material is injected around the ureteral opening to create a valve function and stop urine from flowing back up the ureter.

Around two percent of the population suffers from fecal incontinence, which means loss of the ability to control gases and feces, Solesta® (Oceana Therapeutics) is a biocompatible tissue bulking agent, developed for the treatment of fecal incontinence. It is the only injectable gel to be administered in an outpatient setting without the need for surgery or anesthesia. Solesta is injected in the deep submucosal layer in the proximal part of the anal canal.

All types of incontinence create social embarrassment and a need for new products for non-invasive, less surgical therapies is still present. The present invention proposes a new biocompatible composition to treat such incontinence.

In the field of cosmetic surgery, tissue augmentation is applied for treating aging of the skin. Aging of the skin is one of the most visible changes of the senescence process. In addition, the skin is exposed to numerous factors that are liable to accelerate this physiological process.

Two types of aging of the skin are distinguished. The first is intrinsic (or physiological) aging, which is more easily evaluated on areas of skin that are normally not exposed to sunlight, and the second is extrinsic aging, caused by the interaction of environmental factors, especially UV rays. These environmental factors have a much more pronounced effect on the parts of the body that are exposed to sunlight, especially in the case of people of fair phototype. This is then referred to as actinic aging. Other factors, such as eating habits, smoking, excessive consumption of alcohol, chronic diseases and dysfunction of the endocrine glands, also contribute towards this aging.

During intrinsic aging of the skin, the horny layer is sparingly modified. The epidermis is atrophic and the dermo-epidermal junction is flattened, such that the dermis adheres less well, facilitating the formation of bubbles. The thickness of the dermis is markedly reduced; there are fewer blood vessels. Fewer fibroblasts are also observed, and their capacities for biosynthesis and proliferation are reduced. The elastic fibres first undergo changes, and end up by disappearing.

As regards extrinsic aging, an irregular, occasionally atrophic and occasionally hyperplasic epidermis is observed, with signs of disorganization and dysplasia. The melanocytes are more numerous in certain places, and less numerous in others. There is also irregularity of melanin distribution in the epidermis, following melanosome transfer problems. The number of Langerhans cells decreases. The small blood vessels are first dilated, and then become thin and atrophic.

Wrinkles are the most visible signs of aging. Several types are distinguished, especially surface and deep wrinkles. Deep wrinkles are thought to be due to dermo-hypodermal changes, whereas surface wrinkles are possibly explained by dermal and possibly epidermal changes.

Wrinkles are above all caused by loss of elasticity of the skin. The collagen present in the dermis becomes fragmented, and this fragmentation leads to a loss of structural integrity and to fibroblast dysfunction (Fisher et al., Looking older, Arch Dermatol. 2008; 144(5): 666-672). Impairment of the sub-epidermal elastic network gives rise to surface laxity of aged skin and to folding of its surface. The restructuring of the elastic fibres in the reticular dermis is responsible for the loss of elasticity and of the skin's capacity to resume its shape after stretching. Depending on the type, intensity and topography of the wrinkles, an adapted treatment will be possible.

The skin contains 20% of all the water contained in the human body, and 70% of the skin's water is concentrated in the dermis. It plays a fundamental role by participating in the mechanical properties of the dermis and in the physiological functioning of the skin, especially the integrity of the skin barrier. Hyaluronic acid, which is synthesized by the fibroblasts and the keratinocytes, is a major constituent of the extracellular matrix, which has an important structural function, making it one of the key elements in maintaining the dermal density and thus the firmness of the skin. It is also a veritable water sponge, essential for maintaining moisturization. The synthesis and the quality of hyaluronic acid decrease over the years, causing the dehydration, collapse and loss of firmness of the skin. It is thus fundamental to maintain a good level of moisturization of the skin, in order to limit the aging of the skin.

Certain scars are pathological: among these, mention may be made of hollow scars and relief scars. Hollow scars are hypotrophic scars that may be unsightly, more particularly when they affect the face in the case of acne scars.

The treatment of unaesthetic skin changes related to aging and to scars has made enormous progress in recent years. A relatively large number of natural or synthetic substances have already been described as dermal implants, i.e. as substances injected directly into the skin, in order to remedy skin impairments resulting from aging, trauma or disease.

Such therapeutic techniques are especially the local injection of deactivated botulinum toxin (Botox®) or the use of laser techniques. These various treatments are not exclusive, and their combination has even been recommended. Numerous dermal implants are used at the current time, but none has yet been considered as ideal as a safe and healthy means of tissue increase (Naoum C, Dasiou-Plakida D., Dermal filler materials and botulin toxin, Int. J. Dermatol. October 2001; 40 (10): 609-21).

Given the foregoing, a problem that the invention proposes to solve is that of producing new compositions for tissue augmentation. Preferably, the composition is used for efficiently treating urinary or fecal incontinence, treating aging of the skin, and increasing the durability of filler products, while at the same time promoting their biocompatibility.

The present invention makes it possible to treat urinary or fecal incontinence or to prevent or treat aging of the skin, especially wrinkles and fine lines, with improved efficacy.

One subject of the present invention is thus a combination product comprising both a “mechanical” filler, which is known per se, and a bioresorbable and biodegradable silica-based material. Such a combination makes it possible to be used as a tissue augmentation product to treat incontinence or to treat aging of the skin and scars, especially by filling, efficiently and safely.

A first subject of the invention is thus a combination product comprising:
at least one filler product, and
at least one bioresorbable and biodegradable silica-based material.

The combination product according to the invention is preferentially used for treating incontinence and for treating aging of the skin, and more preferentially for treating wrinkles and fine lines.

The invention also relates to a composition comprising, in a physiologically acceptable medium, at least one filler product and at least one bioresorbable and biodegradable silica-based material.

The composition according to the invention is preferentially administered parenterally for example by injection. Preferentially, the composition according to the invention is injected intradermally, subcutaneously or can be implanted. Preferably, the composition according to the invention is thus in injectable form and administered via a syringe.

The filler product may also contain another active principle, for instance an anaesthetic preferentially such as lidocaine.

The composition is intended for treating aging of the skin and scars. Preferentially, the composition is intended for treating wrinkles and fine lines.

The term “aging of the skin” more particularly means wrinkles, fine lines, fibroblast depletions and dehydration of the skin.

The term “filling product” or “filler” means any product which leads to tissue augmentation and can be administered by injection.

The term composition means pharmaceutical or cosmetical composition, ingredients of which are biocompatible and injectable.

The consistency of the composition of the invention is more or less thick as a function of the depth of the hollow to be filled.

The said filler product is especially chosen from collagen and derivatives thereof, hyaluronic acid, salts thereof and derivatives thereof, alginates, synthetic polymers, elastin and biological polymers, and mixtures thereof.

Preferably, the filler product is chosen from collagen of human origin, collagen of porcine origin, collagen of bovine origin, crosslinked collagens, hyaluronic acid, salts thereof and derivatives thereof, in free or crosslinked form, lactic acid polymers, polycaprolactone polymers, methacrylate derivatives, calcium phosphate derivatives, polyacrylamides, polyurethanes, polyalkylimide gels, polyvinyl microspheres, silicones, silica (SiO₂) polymers and biological polymers, and mixtures thereof.

Collagen is a fibrous protein, of about 300 kDa, which constitutes the connective tissue in the animal kingdom. It may be of human or non-human origin, especially of porcine or bovine origin. Collagen derivatives include, inter alia, crosslinked collagens. Collagen and derivatives thereof may optionally be mixed with an anaesthetic, such as lidocaine.

Wrinkle-filler products based on collagen and derivatives thereof are especially the following:
Cosmoderm® and Cosmoplast® from Inamed/Allergan, which are purified human collagens mixed with lidocaine;
Zyderm® and Zyplast® from Inamed/Allergan, which are highly purified bovine collagens mixed with lidocaine; or alternatively
Evolence® from ColBar, which is composed of crosslinked porcine collagen.

The filler product may also be an alginate or salts thereof. Among the alginates that may be used, mention may be made of sodium alginate, or crosslinked alginates.

The filler product may also be a synthetic polymer. The term “synthetic polymer” means a system formed by an assembly of macromolecules of the same chemical nature, derived from the covalent bonding of a large number of identical or different monomer units of synthetic origin, prepared by polymerization of monomer molecules. Preferably, the said synthetic polymer is chosen from lactic acid polymers such as poly-L-lactic acid, methacrylate derivatives such as hydroxyethyl methacrylate (HEMA), ethyl methacrylate (EMA), polymethyl methacrylate (PMMA), calcium phosphate derivatives such as hydroxyapatite or tricalcium phosphate, polyacrylamides, polyurethanes, polycaprolactone polymers, polyalkylimide gels, polyvinyl microspheres, silicones and silica (SiO₂) polymers, and mixtures thereof.

Wrinkle-filling products based on synthetic polymers are especially the following:
Sculptra® from Dermik, which is a synthetic lactic acid polymer;
Artefill® or Artecoll® from Artes Medical, which is a mixture of PMMA, bovine collagen and lidocaine;
Radiesse® from BioForm, which is calcium hydroxyapatite;
Beta-Altean® from Stiefel, which is tricalcium phosphate;
Bio-Alcamid® from SkinRx Distribution Inc., which is a polyalkylimide gel, or alternatively
Evolution® from Procytech SA, which is polyvinyl microspheres, or alternatively
Aquamid® from Ferrosan A/S/Contura, which is a polyacrylamide gel.

The filler product may also be a biological polymer. The term “biological polymer” means a system formed by an assembly of macromolecules of the same chemical nature, derived from the covalent bonding of a large number of identical or different monomer units of natural or artificial origin (obtained by chemical modification of a natural polymer). Preferably, the said biological polymer is chosen from methylcellulose, agarose, dextran and chitosan polymers. Wrinkle-filling products based on biological polymers are especially the following:
Reviderm Intra® from Rofil Medical International, which is a dextran polymer.

More preferentially, the filler product is chosen from hyaluronic acid, a pharmaceutically acceptable salt or a derivative thereof, particularly a sodium or potassium salt. Hyaluronic acid may be used in various forms: a salt, a derivative such as an ester or an amide, in linear or crosslinked form. In particular, the molecular weight is conventionally between 500 kDa and 5000 kDa and the degree of crosslinking depends on the use and the place of application, especially in the field of wrinkles. Various crosslinking agents are used, such as oxides, polyaziridyl compounds or glycidyl ethers, and more specifically 1,4-butanediol diglycidyl ether.

The term “pharmaceutically acceptable salt” means a basic or acidic salt, of which non-limiting examples include hydrochloride, hydrobromide, hydriodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylenebis(2-hydroxy-3-naphthoate)), and aluminium, calcium, lithium, magnesium, potassium, sodium, zinc or diethanolamine salts. Certain compounds according to the invention may form pharmaceutically acceptable salts with various amino acids (Berge et al., 66 J. Pharm. Sci. 1-19 (1977)).

Hyaluronic acid is a ubiquitous natural polysaccharide that exists in the same form from the simplest bacterium to man. It is a polysaccharide composed alternately of D-glucuronic acid and N-acetylglucosamine linked together via alternating beta-1,4 and beta-1,3 glycoside bonds. According to Saari H et al. (Differential effects of reactive oxygen species on native synovial fluid and purified human umbilical cord hyaluronate, Inflammation 17 (1993): 403-415), polymers of this repeating unit may have a size of between 10² and 10⁴ kDa in vivo, hyaluronic acid collected from umbilical cord having a weight of 2500 kDa.

Hyaluronic acid is a natural constituent of the dermis, where it plays an important role in the moisturization and elasticity of the skin. However, it decreases in quantity and quality with age, leading to drying-out of the skin, which becomes wrinkled. It is highly water-soluble and forms solutions of high viscosity in water. The tolerance of hyaluronic acid is very good and no immunogenicity is associated with this substance.

Hyaluronic acid may be of human or non-human origin, for instance of avian or bacterial origin.

Hyaluronic acid may also be combined with at least one dextran, in order to slow down its in vivo degradation.

Wrinkle-filling products based on hyaluronic acid and derivatives thereof are especially the following:
Restylane® and Perlane® from Galderma/Q-Med, or Juvéderm® from Allergan/Corneal, or Prevelle Silk® from Genzyme, which are hyaluronic acids from Streptococcus bacteria;
Hylaform®, Hylaform Plus® or Captique® from Genzyme/Allergan, which are chemically modified hyaluronic acids of avian origin;
Elevess® from Anika, which is composed of hyaluronic acid from Streptococcus bacteria mixed with lidocaine; or alternatively
Matridex® from TBMC Aesthetics, which is a gel composed of hyaluronic acid combined with dextran beads (DEAE Sephadex).
The Emervel® filler range from Galderma, which proposes five products based on hyaluronic acid, with or without lidocaine, of entirely different textures, with different degrees of crosslinking of the HA and different gel particle sizes, each with its specific indications.

Incontinence products based on hyaluronic acid and derivatives are especially the following:
Deflux sold by Oceana Therapeutics, is a sterile, highly viscous gel of dextranomer microspheres in a carrier gel of non-animal stabilized hyaluronic acid.
Solesta® sold by Oceana Therapeutics is a biocompatible tissue bulking agent, consisting of dextranomer microspheres and stabilized sodium hyaluronate.

According to the present invention, the filler product preferentially used is hyaluronic acid.

The hyaluronic acid can be obtained from various sources of animal and non-animal origin. Sources of non-animal origin include yeast and preferably bacteria. The molecular weight of a single hyaluronic acid molecule is typically in the range of 0.1-10 MDa, but other molecular weights are possible. In certain embodiments the concentration of said hyaluronic acid is in the range of 1 to 100 mg/ml. In some embodiments the concentration of said hyaluronic acid is in the range of 2 to 50 mg/ml. In specific embodiments the concentration of said hyaluronic acid is in the range of 5 to 30 mg/ml or in the range of 10 to 30 mg/ml.

In certain embodiments, the hyaluronic acid is crosslinked. Crosslinked hyaluronic acid comprises crosslinks between the hyaluronic acid chains, which creates a continuous network of hyaluronic acid molecules which is held together by the covalent crosslinks, physical entangling of the hyaluronic acid chains and various interactions, such as electrostatic interactions, hydrogen bonding and van der Waals forces. Crosslinking of the hyaluronic acid may be achieved by modification with a chemical crosslinking agent. The chemical crosslinking agent may for example selected from the group consisting of divinyl sulfone, multiepoxides and diepoxides. According to embodiments the chemical crosslinking agent is selected from the group consisting of 1,4-butanediol diglycidyl ether (BDDE), 1,2-ethanediol diglycidyl ether (EDDE) and diepoxyoctane. According to a preferred embodiment, the chemical crosslinking agent is 1,4-butanediol diglycidyl ether (BDDE).

The crosslinked hyaluronic acid product is preferably biocompatible. This implies that no, or only very mild, immune response occurs in the treated individual. That is, no or only very mild undesirable local or systemic effects occur in the treated individual.

The crosslinked hyaluronic acid product according to the invention may be a gel, or a hydrogel. That is, it can be regarded as a water-insoluble, but substantially dilute crosslinked system of hyaluronic acid molecules when subjected to a liquid, typically an aqueous liquid.

The gel contains mostly liquid by weight and can e.g. contain 90-99.9% water, but it behaves like a solid due to a three-dimensional crosslinked hyaluronic acid network within the liquid. Due to its significant liquid content, the gel is structurally flexible and similar to natural tissue, which makes it very useful as a scaffold in tissue engineering and for tissue augmentation.

As mentioned, crosslinking of hyaluronic acid to form the crosslinked hyaluronic acid gel may for example be achieved by modification with a chemical crosslinking agent, for example BDDE (1,4-butandiol diglycidylether). The hyaluronic acid concentration and the extent of crosslinking affects the mechanical properties, e.g. the elastic modulus G′, and stability properties of the gel. Crosslinked hyaluronic acid gels are often characterized in terms of “degree of modification”. The degree of modification of hyaluronic acid gels generally range between 0.1 and 15 mole %. The degree of modification (mole %) describes the amount of crosslinking agent(s) that is bound to HA, i.e. molar amount of bound crosslinking agent(s) relative to the total molar amount of repeating HA disaccharide units. The degree of modification reflects to what degree the HA has been chemically modified by the crosslinking agent. Reaction conditions for crosslinking and suitable analytical techniques for determining the degree of modification are all well known to the person skilled in the art, who easily can adjust these and other relevant factors and thereby provide suitable conditions to obtain a degree of modification in the range of 0.1-2% and verify the resulting product characteristics with respect to the degree of modification. A BDDE (1,4-butandiol diglycidylether) crosslinked hyaluronic acid gel may for example be prepared according to the method described the published international patent application WO 9704012, incorporated herein.

In a preferred embodiment the hyaluronic acid of the composition is present in the form of a crosslinked hyaluronic acid gel crosslinked by a chemical crosslinking agent, wherein the concentration of said hyaluronic acid is in the range of 10 to 30 mg/ml and the degree of modification with said chemical crosslinking agent is in the range of 0.1 to 2 mole %.

Hyaluronic acid gels may also comprise a portion of hyaluronic acid which is not crosslinked, i.e not bound to the three-dimensional crosslinked hyaluronic acid network. However, it is preferred that at least 50% by weight, preferably at least 60% by weight, more preferably at least 70% by weight, and most preferably at least 80% by weight, of the hyaluronic acid in a gel composition form part of the crosslinked hyaluronic acid network.

According to the present invention, the composition also comprises a bioresorbable and biodegradable silica-based material. Various efforts are still under way to develop novel biodegradable materials and/or bioresorbable materials for various applications in human medicine and in medical technology. These sectors moreover exert increasingly high demands, in particular as regards the biocompatibility, the biological activity and the toxicological properties of the materials.

The expression “bioresorbable and biodegradable” refers to a product which has a resorption time of between 5 and 10 years following injection preferentially between 2 and 5 years and more preferentially in the year following injection.

In a specific embodiment according to the invention, when used for treating incontinence, preferably the composition of the invention will have a resorption time of between 5 and 10 years, In another specific embodiment according to the invention, when used for treating skin aging, preferably the composition of the invention will have a resorption time of a year following injection.

Absorbable silica gels are known in the prior art. DE 196 09 551 especially describes biodegradable, bioresorbable silica fibres, but the process does not make it possible to obtain good cytotoxicity or an optimum production yield for these fibres.

The bioresorbable and biodegradable silica-based material that may be used according to the present invention is preferentially a non-cytotoxic material as described in patent application CA 2675181, which is incorporated in its entirety into the present patent application.

More particularly, the material used in the composition according to the invention is a silica gel, particles or fibres, as described in the abovementioned patent application. The silica-basedl material and the products derived from the process according to the invention described in CA 2675181 may be prepared in the form of particles, filaments, fibres, fibrous non-woven and/or woven laps, and have excellent biodegradability and bioabsorbability. Another advantage of the silica fibres according to CA 2675181 is that they have improved biocompatibility by means of the described production processes. Moreover, it has been shown experimentally that the claimed nonwoven silica fibres and fibrous networks have better wound-cicatrizing properties. More particularly, these materials may thus be used advantageously in the wound and scar treatment sector.

The Applicant has discovered that a bioresorbable and biodegradable combination of a filler product and of a silica-based material can improve the degradation time of the filler after injection, while at the same time maintaining excellent biocompatibility. Furthermore, the injection of foreign substances into the cutaneous matrix makes it possible to induce neocollagen synthesis. Thus, the injectable composition according to the invention makes it possible to treat aging of the skin and scars, and more preferentially to treat wrinkles and fine lines.

Preferentially the silica-based material is a silica gel, fibers or particles. More preferably, the silica-based material is in the form of particles, ranging in size between 50-250 microns, preferably between 80 and 150 microns, with an average preferable size of about 100 microns. In a specific embodiment, the stabilized hyaluronic acid acts mainly as a carrier, leaving the silica-based particles at the implant site.

The composition according to the invention comprises the silica-based compound in a concentration between 10 and 200 mg/g per weight of the composition. Preferentially, the silica-based material is present in a range between 40 and 130 mg/g of the total composition.

In a known manner, the compositions according to the invention may also contain common adjuvants that are well known to those skilled in the art. In particular, the composition according to the invention may also contain another active principle. Preferably the composition contains, for instance an anaesthetic selected from the group consisting of amide and ester type local anesthetics or a combination thereof. A local anesthetic is a drug that causes reversible local anesthesia and a loss of nociception. When it is used on specific nerve pathways (nerve block), effects such as analgesia (loss of pain sensation) and paralysis (loss of muscle power) can be achieved. The local anesthetic may be added to the hyaluronic acid composition to reduce pain or discomfort experienced by the patient due to the injection procedure. The groups of amide (also commonly referred to as aminoamide) type local anesthetics and ester (also commonly referred to as aminoester) type local anesthetics are well defined and recognized in the art.

Amide and ester type local anesthetic molecules are built on a simple chemical plan, consisting of an aromatic part linked by an amide or ester bond to a basic side-chain. The only exception is benzocaine which has no basic group. All other anesthetics are weak bases, with pKa values mainly in the range 8-9, so that they are mainly but not completely, ionized at physiological pH. As a result of their similarity they may be expected to have similar chemical and physical effects on the hyaluronic acid composition.

According to certain embodiments the local anesthetic is selected from the group consisting of amide and ester type local anesthetics, for example bupivacaine, butanilicaine, carticaine, cinchocaine (dibucaine), clibucaine, ethyl parapiperidinoacetylaminobenzoate, etidocaine, lignocaine (lidocaine), mepivacaine, oxethazaine, prilocaine, ropivacaine, tolycaine, trimecaine, vadocaine, articaine, levobupivacaine, amylocaine, cocaine, propanocaine, clormecaine, cyclomethycaine, proxymetacaine, amethocaine (tetracaine), benzocaine, butacaine, butoxycaine, butyl aminobenzoate, chloroprocaine, dimethocaine (larocaine), oxybuprocaine, piperocaine, parethoxycaine, procaine (novocaine), propoxycaine, tricaine or a combination thereof.

According to some embodiments the local anesthetic is selected from the group consisting of bupivacaine, lidocaine, and ropivacaine, or a combination thereof. According to specific embodiments the local anesthetic is lidocaine. Lidocaine is a well-known substance, which has been used extensively as a local anesthetic in injectable formulations, such as hyaluronic acid compositions.

The concentration of the amide or ester local anesthetic may be selected by the skilled person within the therapeutically relevant concentration ranges of each specific local anesthetic or a combination thereof. In certain embodiments the concentration of said local anesthetic is in the range of 0.1 to 30 mg/ml. In some embodiments the concentration of said local anesthetic is in the range of 0.5 to 10 mg/ml. When lidocaine is used as the local anesthetic, the lidocaine may preferably be present in a concentration in the range of 1 to 5 mg/ml, more preferably in the range of 2 to 4 mg/ml, such as in a concentration of about 3 mg/ml.

In a more specific embodiment, there is provided an injectable hyaluronic acid composition, in the form of a gel, comprising: an aqueous hyaluronic acid gel comprising 2 to 50 mg/ml of a hyaluronic acid; 0.5 to 10 mg/ml of lidocaine; and 40 to 130 mg/g of silica-based particles.

The composition of the invention is preferably administered parenterally. The term “parenterally” means subcutaneous or intradermal application. As non-limiting examples of parenteral compositions, mention may be made of compositions in the form of solutions or suspensions for perfusion or for injection. Via the parenteral route, the compositions may be administered via standard syringes or double syringes (in which two compositions are separated from each other by a leaktight membrane, and in which mixing takes place on exiting the syringe). Man skilled in the art will adapt the size of the needle of the syringe to the composition according to the invention. Needles can be chosen from the range of size between 15 and 30 G, preferably between 21 G and 27 G.

The composition according to the invention, may be provided in the form of a pre-filled syringe, i.e. a syringe that is pre-filled with the composition of the invention and autoclaved.

In some embodiments, the composition has been subjected to sterilization. In certain embodiments is the composition sterilized, i.e. the composition has been subjected to heat and/or steam treatment in order to sterilize the composition. In some embodiments the composition has been subjected to sterilization by autoclaving or similar sterilization by heat or Steam.

The invention relates to a composition comprising at least one-filler product and at least one bioresorbable and biodegradable silica-based material, for treating aging of the skin and scars. The composition according to the invention is suitable for treating wrinkles and/or aged skin, and is especially directed towards reducing the effects thereof. The treatment of wrinkles, fine lines, fibroblast depletions, skin dehydration and scars of all types is especially performed by filling.

The composition according to the invention may be applied to facial and bodily skin. In particular, the composition according to the invention may be applied to areas of the face or the forehead that are marked with expression wrinkles or with scars, caused, inter alia, by acne.

The invention also relates to the use of a composition comprising at least one wrinkle-filling product and at least one bioresorbable and biodegradable silica-based material, to be used in repair surgery.

The invention concerns a combination product or composition for treating aging of the skin.

The invention also related to the use of a combination product or composition to treat aging of the skin and scars, preferably to treat wrinkles.

The invention also concerns a combination product or a composition for treating fecal or urinary incontinence. The invention also relates to the use of a composition comprising at least one filling product and at least one bioresorbable and biodegradable silica-based material, to treat fecal or urinary incontinence.

Without desiring to be limited thereto, the present invention will in the following be illustrated by way of examples.

### EXAMPLE 1

### Composition According to the Invention

Spherical silica particles were mixed with HA gel and evaluated regarding the possibility to inject into ex vivo tissue. The silica particles are solid particles whereas the HA gel particles are soft. The evaluation showed that it was possible to inject a mixture of silica particles and gel particles in ex vivo tissue.

Two gel prototypes have been manufactured mixing cross-linked HA gel with spherical silica particles.

Materials used in the study is displayed in the below table. The gel prototypes were filled in 1 mL glass syringes. Before injection, the syringes were centrifuged to remove air in the formulation.

| Material | Information |
|---|---|
| Gel prototype 1; 47 mg SiO₂/g gel | Batch No 1 |
| Gel prototype 2; 128 mg SiO₂/g gel | Batch No 2 |
| Reference gel | HA gel crosslinked with BDDE |
| Syringe | BD, 1 mL glass syringe |
| Injection needle | BD 30 G |
| | BD 27 G |
| | Braun 25 G |
| | BD 23 G |
| | Braun 21 G 0.8 × 50 mm |
| | BD 18 G |
| Ex vivo tissue | Female donor, abdominal skin |

### EXAMPLE 2

### Visualisation of the Gel Composition of Example 1 According to the Invention

The gel prototypes were evaluated and photographed in two different microscopes, a Leica DM500 and a stereomicroscope Leica MZ16 A.

Leica DM500

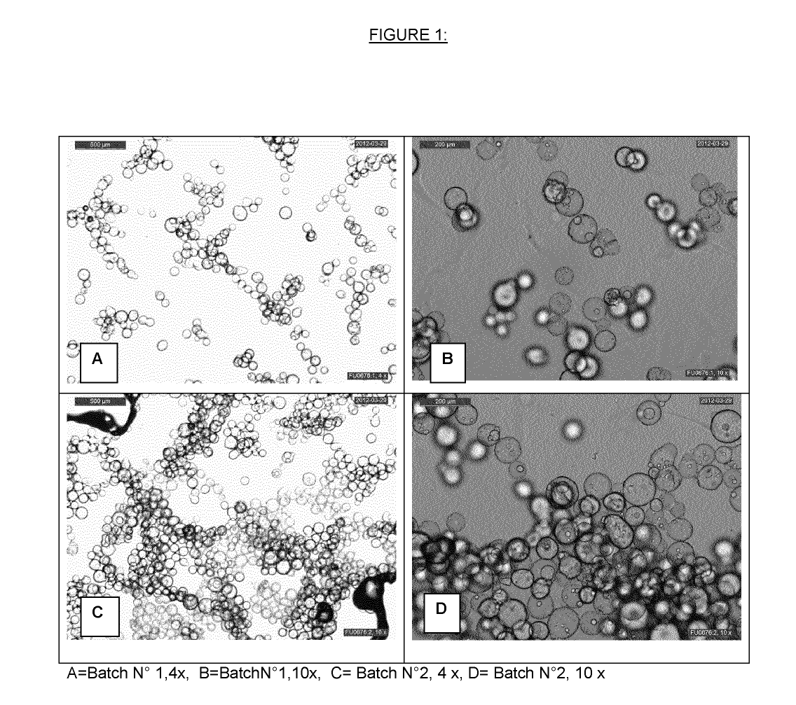

A small drop of gel prototype was put on a microscope slide and covered with a cover glass. The cover grass was pressed to form a monolayer of silica particles. Photos were taken with 4× and 10× objectives and eyepieces were 10×. Example pictures are displayed below. The scale bar shows the approximate size of the silica particles. The HA gel particles are not visible. The silica particles are circular, See FIG. 1.

Leica MZ 16 A

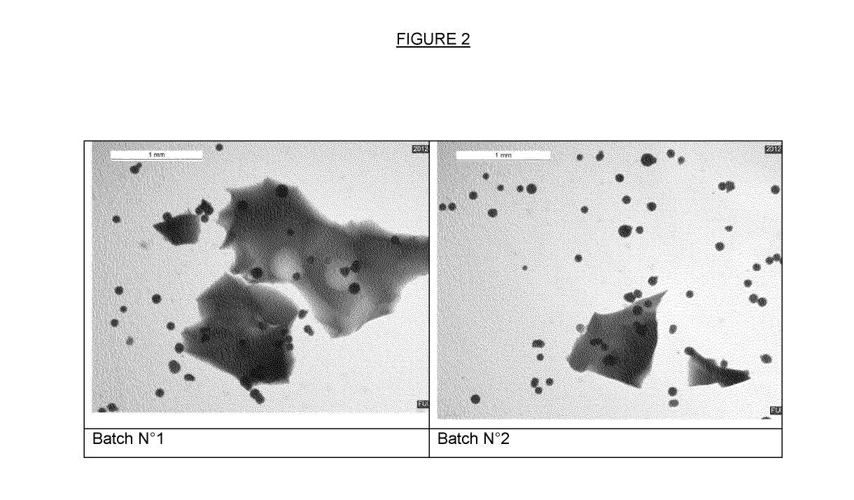

The gel prototypes were coloured with toluidine blue. Both the silica particles and the gel particles were stained, see FIG. 2. Measurements of the size of silica particles showed a mean diameter of approximately 100 μm. A maximum size of a single particle was found to be approximately 150 μm in diameter. The photos are chosen to visualize the two types of particles in the gel prototypes, the particles of HA gel and the spherical particles of silica-based material.

### EXAMPLE 3

### Injection into Human Ex Vivo Abdominal Tissue

The human abdominal skin was dissected until approximately 2 cm of fat tissue remained. The skin was pinned to a cork plate. The skin was marked with the test material identification and injections performed underneath the respective identification. Cross-linked HA gel alone was used as reference gel. The test material was coloured with green food dye to be able to be visualized in the tissue and filled in 1 mL glass syringes. The injection needle chosen for the injections was the Braun 21 G. The test materials were injected into the deep layers of dermis but predominantly into the subcutis layer. Approximately 0.3 mL was injected at the same spot.

The gel prototypes were both easily palpable as harder,material while the HA gel of reference was more soft.

The skin was then dissected for visualization of the gel inside the tissue.

It was possible to inject two different concentrations of silica particles mixed with a HA gel in ex vivo tissue. The injection depth and injection needle should be chosen with respect to the intended use.

1-21. (canceled)

22. Combination product comprising at least one filler product, which is cross-linked hyaluronic acid or a derivative thereof, and at least one bioresorbable and biodegradable silica-based material in the form of particles.

23. Combination product according to claim 22, wherein the particles of the bioresorbable and biodegradable silica-based material are ranging in size between 50 and 250 microns.

24. Combination product according to claim 22, wherein the cross-linked hyaluronic acid filler product is in the form of particles.

25. Combination product according to claim 22, wherein the combination product is further comprising an anesthetic.

26. Combination product according to claim 25, wherein the anesthetic is lidocaine.

27. Composition comprising, in a physiologically acceptable medium, a combination product according to claim 22.

28. Composition according to claims 27, wherein the composition comprises 40 to 130 mg/g of the silica-based material in the form of particles.

29. Composition according to claim 27, wherein the composition comprises 2 to 50 mg/ml of cross-linked hyaluronic acid.

30. Composition according to claim 28, wherein the composition comprises 2 to 50 mg/ml of cross-linked hyaluronic acid.

31. Composition according to claim 27, wherein the composition comprises 1 to 5 mg/ml of lidocaine.

32. A method of augmenting tissue comprising administering the combination product according to claim 22.

33. The method according to claim 32, wherein the combination product is administered parenterally.

34. A method of treating aging of the skin and scars comprising administering the combination product according to claim 22.

35. The method according to claim 34, wherein the treatment of aging of the skin comprises the treatment of wrinkles, fine lines, fibroblast depletions and dehydration of the skin.

36. The method according to claim 34, wherein the combination product is administered parenterally.

37. A method of treating fecal or urinary incontinence comprising administering the combination product according to claim 22.

38. The method according to claim 37, wherein the combination product is administered parenterally.

39. A method of augmenting tissue comprising administering the composition according to claim 27.

40. The method according to claim 39, wherein the composition is administered parenterally.

41. A method of treating aging of the skin and scars comprising administering the composition according to claim 27.

42. The method according to claim 41, wherein the treatment of aging of the skin comprises the treatment of wrinkles, fine lines, fibroblast depletions and dehydration of the skin.

43. The method according to claim 41, wherein the composition is administered parenterally.

44. A method of treating fecal or urinary incontinence comprising administering the composition according to claim 27.

45. The method according to claim 44, wherein the composition is administered parenterally.
